(19) United States
(12) Patent Application Publication
(10) Pub. No.: US 2020/0022949 A1
(43) Pub. Date: Jan. 23, 2020

(54) COMPOSITION AND METHOD FOR THE ALLEVIATION OF EFFECTS OF ALCOHOL CONSUMPTION

(71) Applicant: Detoxicated Inc., San Diego, CA (US)

(72) Inventor: Jennifer Davis, San Diego, CA (US)

(21) Appl. No.: 16/516,167

(22) Filed: Jul. 18, 2019

(30) Foreign Application Priority Data
Jul. 20, 2018 (CA) 3012021

(51) Int. Cl. A61K 31/353; A61K 31/197; A61K 47/44; A61K 47/55; A61K 31/4415; A61K 33/32; A61K 36/33; A61K 31/351; A61K 33/06; A61K 33/30; A61K 33/04; A61K 31/185

(52) U.S. Cl. CPC A61K 31/353; A61K 31/197; A61K 47/44; A61K 47/551; A61K 31/4415; A61K 31/185; A61K 36/33; A61K 31/351; A61K 33/06; A61K 33/30; A61K 33/04; A61K 33/32

(57) ABSTRACT

A composition for alleviating hangover symptoms in a subject in need thereof. The composition comprises dihydromyricetin, N-Acetyl Cysteine, taurine, salicin, niacin, vitamin B6, selenium, manganese and prickly pear powder. The composition can be formulated as pills or capsules. The dihydromyricetin component may be coated with soy lecithin to maximize absorption. The composition includes a first composition and a separate second composition. The composition is administered as a first pill or capsule comprising the first composition prior to alcohol consumption and a second pill comprising the first composition taken after alcohol consumption.

### PRIORITY CLAIM

This application claims the benefit, under the Paris Convention, of Canadian Patent Application No. 3,012,021, filed Jul. 20, 2018, the contents of which are incorporated herein by this reference.

### TECHNICAL FIELD

The disclosure relates to compositions and methods for preventing, alleviating and treating symptoms and effects of alcohol consumption.

### BACKGROUND

A number of negative physiological effects can follow the consumption of alcohol. More pronounced negative effects are known as a hangover. Effects from alcohol consumption typically manifest several hours after consumption of alcohol. Symptoms experienced following alcohol consumption include headache, drowsiness, concentration problems, dry mouth, dizziness, fatigue and nausea. The most pronounced effects experienced following over consumption of alcohol are headache and nausea. A severe hangover can be incapacitating leading to economic loss for both employees and employers.

It is generally known that a number of precautions can be taken contemporaneously with alcohol consumption and after in order to mitigate the risk of suffering from deleterious effects following alcohol consumption. For example, due the dehydrating effect of alcohol, it is recommended to drink copious of amounts of water when consuming alcohol and following consumption. It is also well known that consuming food with alcohol can mitigate intoxication and reduce the potential for any number of negative effects including a hangover following alcohol consumption. Some recommend taking anti pain medications such as ibuprofen or aspirin following alcohol consumption in order to avoid the onset of the headache hangover symptom following alcohol consumption.

A number of non-scientifically proven hangover remedies have gained popularity at various times such as mixtures including raw eggs, tabasco sauce and tomato juice. Various hangover remedies in the form of nutritional supplements have also been introduced in recent years. There is a need for a composition for alleviating the negative effects of an alcohol consumption including hangover symptoms that is safe, effective and relatively inexpensive. There is a need for such a composition that additionally promotes general health and well-being. There is a further need for a composition that can be easily administered in form of a pill or capsule and that targets specific needs during all phases of alcohol metabolism.

### BRIEF SUMMARY

A composition for alleviating alcohol consumption symptoms including hangover symptoms in a subject in need thereof is provided that includes dihydromyricetin (DHM), N-Acetyl Cysteine (NAC) and a number of co-factors. The composition comprises soy lecithin. The composition is preferably formulated as pills or capsules.

A method for alleviating alcohol consumption symptoms including hangover symptoms in a subject in need thereof is provided that includes the steps of administering a first composition to the subject prior to alcohol consumption and then delivering a second composition to the subject after alcohol consumption. The first composition is preferably provided in the form of two pills or capsules prior to alcohol consumption. The second composition is preferably provided in the form of two pills or capsules after alcohol consumption.

According to one aspect of the disclosure, there is provided a composition for alleviating alcohol consumption symptoms including hangover symptoms in a subject in need thereof, the composition comprising dihydromyricetin and N-Acetyl Cysteine, wherein the composition is formulated as pills or capsules.

According to another aspect of the disclosure, there is provided a composition for alleviating alcohol consumption symptoms including hangover symptoms in a subject in need thereof, the composition comprising dihydromyricetin, N-Acetyl Cysteine and soy lecithin.

According to another aspect of the disclosure, there is provided a composition for alleviating alcohol consumption symptoms including hangover symptoms in a subject in need thereof comprising dihydromyricetin, N-Acetyl Cysteine, soy lecithin, zinc, selenium, magnesium, manganese, taurine, prickly pear powder, niacin, vitamin B6 and salicin.

According to another aspect of the disclosure, there is provided a composition for alleviating alcohol consumption symptoms including hangover symptoms in a subject in need thereof. The composition includes a first composition and a separate second composition. The first composition comprises dihydromyricetin, N-Acetyl Cysteine, soy lecithin and prickly pear powder. The second composition comprises dihydromyricetin, N-Acetyl Cysteine, soy lecithin and salicin.

According to another aspect of the disclosure, there is provided a composition for alleviating alcohol consumption symptoms including hangover symptoms in a subject in need thereof. The composition includes a first composition and a separate second composition. The first composition comprises from about 20% to about 30% by weight of dihydromyricetin, from about 28% to about 38% by weight of N-Acetyl Cysteine, and soy lecithin. The second composition comprises from about 45% to about 55% by weight of dihydromyricetin, from about 18% to about 28% by weight of N-Acetyl Cysteine, and soy lecithin.

According to yet another aspect of the disclosure, there is provided a method of alleviating alcohol consumption symptoms including hangover symptoms in a subject in need thereof comprising the steps of administering a first composition comprising dihydromyricetin, N-Acetyl Cysteine and prickly pear powder to the subject prior to alcohol consumption and then administering a second composition comprising dihydromyricetin and N-Acetyl Cysteine and salicin to the subject after alcohol consumption.

According to another aspect of the disclosure, there is provided a use of a composition for alleviating alcohol consumption symptoms including hangover symptoms in a subject in need thereof. The composition includes a first composition and a separate second composition. The first composition comprises from about 20% to about 30% by weight of dihydromyricetin, from about 28% to about 38% by weight of N-Acetyl Cysteine, and soy lecithin. The second composition comprising from about 45% to about 55% by weight of dihydromyricetin, from about 18% to about 28% by weight of N-Acetyl Cysteine, and soy lecithin.

### DETAILED DESCRIPTION

The present composition for alleviating alcohol consumption symptoms including hangover symptoms includes dihydromyricetin (DHM), which is also known as ampelopsin. It is a flavonoid found in plants, such as vine tea or moyeam tea. DHM enhances the body's natural ability to break down acetaldehyde, the alcohol byproduct responsible for most hangover symptoms. In addition, DHM protects the liver by reducing oxidative stress and lowering liver enzyme elevation in animal studies. DHM possesses anti-inflammatory effects that serve to protect the liver, not only during alcohol consumption. In a human study conducted by Chen et al. (2015), DHM was shown to improve liver health in patients with non-alcoholic fatty liver disease by decreasing levels of liver enzymes and inflammatory cytokines, as increases in either can indicate liver disease.

In animal models, DHM has been shown to counteract the neurotransmitter changes that results in poor sleep and anxiety following alcohol consumption. DHM has shown much versatility in its health benefits in the past decade, demonstrating that it possesses anti-cancer, anti-inflammatory, and antioxidant properties, along with improving metabolic conditions, such as diabetes mellitus, and protects against neurodegenerative diseases. DHM has been shown to reduce the likelihood and severity of hangover symptoms. Also, DHM has been shown to protect the liver by preventing liver enzyme elevation, which indicates liver inflammation in animal studies.

The source of DHM for the present composition is preferably vine tea. Vine tea, also known as longevity tea, moyeam tea or Ampelopsis grossedentata, has been brewed by countries in Eastern Asia as they believed the stems and leaves of the plant prevented hangovers, cured diseases and promoted a long life. Vine tea has a high density of amino acids, trace elements, and contains the greatest amounts of flavonoids among all plants causing it to display potent antioxidant properties. Studies have also shown that vine tea possesses anti-bacterial properties, improves immune function, decreases blood sugars and fats, and protects the liver.

Alternatively, the source of DHM can be Hovenia dulcis, which is also known as the fruit of oriental raisin tree, Japanese raisin tree, or Chinese raisin tree.

DHM is preferably provided in the form of a powder in the composition of the disclosure as the powder form retains the structure of DHM and prevents decomposition through exposure to light, thus improving DHM stability and improving efficacy. Unlike many prior art products, the composition of the disclosure is preferably provided in individual sealed serving packets that retain a fresh quality of the ingredients by preventing exposure to air.

The DHM is preferably coated with soy lecithin prior to addition into the capsule or pill in order to improve bioavailability. Alternatively, the DHM and the soy lecithin may be mixed together.

The present composition also includes N-Acetyl Cysteine (NAC), which is an antioxidant that is well-known for its detoxification benefits. N-Acetyl Cysteine neutralizes free radicals by replenishing glutathione, an antioxidant naturally found in the human body. Although levels of glutathione are quickly depleted when metabolizing alcohol, supplementing with NAC can ensure that the human body has the dietary components critical to proper alcohol metabolism. NAC replenishes natural antioxidant stores to eliminate toxic by-products produced by alcohol. NAC is known to oxidize when exposed to open air. Given that the composition of the disclosure is preferably provided in sealed in individual packets, oxidation of NAC is prevented.

Additional key ingredients of the present composition include taurine, prickly pear, and salicin.

Taurine contributes to the benefits of the present composition by enhancing the enzymes involved in alcohol metabolism and in detoxification. In order for all these enzymes to function, important cofactors are required, such as zinc, selenium, magnesium and manganese.

Prickly pear is preferably provided in the form of a powder. Prickly pear extract reduces the inflammation that causes the hangover.

Salicin is an anti-inflammatory. It is known that higher levels of inflammation are associated with alcohol consumption. The source of salicin is preferably white willow extract. White willow extract from the bark of the white willow tree is a potent anti-inflammatory used in naturopathic medicine. Studies have shown that high levels of inflammation after alcohol consumption are associated with increased hangover symptoms. White willow extract works similarly to aspirin and can help mitigate hangover symptoms caused by inflammation.

The composition includes a number of supporting ingredients. These ingredients include zinc, selenium, magnesium, manganese, niacin and vitamin B6.

The composition preferably includes a first composition and a separate second composition. The first composition and the second compositions are preferably formulated as separate pills or capsules. The first composition is preferably in the form of a first pill or capsule, and is preferably taken before alcohol consumption. According to a preferred embodiment of the disclosure, the first pill or capsule preferably has the following components and relative amounts as set out in Table 1.

TABLE 1

| Component | Amount (per two capsules or pills) | |
|---|---|---|
| Niacin | 15 | mg |
| Vitamin B6 (as Pyridoxine HCl) | 50 | mg |
| Magnesium (as Magnesium Oxide) | 50 | mg |
| Zinc (as Carnosine) | 5 | mg |
| Selenium (as L-Selenomethionine) | 100 | mcg |
| Manganese (as Manganese Citrate) | 2 | mg |
| N-Acetyl Cysteine | 400 | mg |
| DHM (Vine Tea Extract) | 300 | mg |
| Soy Lecithin Powder | 50 | mg |
| Prickly Pear Powder | 150 | mg |
| Taurine | 100 | mg |

The first pill or capsule is preferably white in color. Preferably, two of the first composition pills or capsules are taken before alcohol consumption.

Alternatively, the first pill or capsule may have following components and relative amounts as set out in Table 2.

TABLE 2

| Component | Amount (per two capsules or pills) | |
|---|---|---|
| Niacin | 25 | mg |
| Vitamin B6 (as Pyridoxine HCl) | 75 | mg |
| Magnesium (as Magnesium Oxide) | 50 | mg |
| Zinc (as Carnosine) | 5 | mg |
| Selenium (as L-Selenomethionine) | 100 mcg to 200 mcg | |
| Manganese (as Manganese Citrate) | 2 | mg |
| N-Acetyl Cysteine | 400 | mg |
| DHM (Vine Tea Extract) | 100 mg to 300 mg | |
| Soy Lecithin Powder | 50 | mg |
| Prickly Pear Powder | 150 | mg |
| Taurine | 100 | mg |

The second composition is preferably in the form of a second pill or capsule. The second pill or capsule is preferably taken after alcohol consumption. According to the preferred embodiment of the disclosure, the second pill or capsule preferably has the following components and relative amounts as set out in Table 3.

TABLE 3

| Component | Amount (per two capsules or pills) | |
|---|---|---|
| Zinc (as Zinc Gluconate) | 5 | mg |
| Selenium (Selenomethionine) | 100 | mcg |
| Manganese (as Manganese Citrate) | 2 | mg |
| N-Acetyl Cysteine | 300 | mg |
| DHM (Vine Tea Extract) | 650 | mg |
| Soy Lecithin Powder | 50 | mg |
| Salicin (from White Willow) | 120 | mg |
| Taurine | 100 | mg |

Alternatively, the second pill or capsule may have following components and relative amounts as set out in Table 4.

TABLE 4

| Component | Amount (per two capsules or pills) | |
|---|---|---|
| Zinc (as Zinc Gluconate) | 5 | mg |
| Selenium (Selenomethionine) | 100 mcg to 200 mcg | |
| Manganese (as Manganese Citrate) | 2 | mg |
| N-Acetyl Cysteine | 400 | mg |
| DHM (Vine Tea Extract) | 500 | mg |
| Soy Lecithin Powder | 50 | mg |
| Salicin (from White Willow) | 120 | mg |
| Taurine | 100 | mg |

The second pill or capsule is preferably green in color. Preferably, two of the second composition pills or capsules are taken after alcohol consumption.

In both of the first and second pills or capsules, the N-Acetyl Cysteine, DHM and soy lecithin powder are provided in the form of a blend.

The first and second pills or capsules preferably additionally comprise methylcellulose. However, other fillers and/or flow agents known in the art may also be employed in the place of methylcellulose. Where the composition is formulated as a capsule, the capsules preferably comprise gelatin.

The combination of the first composition pills or capsules preferably taken before alcohol consumption and the second composition pills or capsules preferably taken after alcohol consumption targets specific needs during all phases of alcohol metabolism. Each ingredient of the pills or capsules maximizes the benefits of hangover alleviation. Alternatively, both of the first and second pills or capsules can be taken at the same time after alcohol consumption.

Alternative routes of administration of the composition of the disclosure include administering the composition in other forms including a powder, liquid, 2-piece encapsulation, various pills with carrier, binders, effervescent drink, chewing gums, time-released capsules, rapid-release capsules, nanoparticulation of the ingredients, energy shots, liquid and soft-gel capsules.

### Example 1—Administration of Before and after Formulation

The administration of two capsules having the components listed in Table 1 were administered to 100 subjects prior to consumption of at least five alcoholic beverages containing at least 1.5 ounces of alcohol within two hours. Two capsules having the components listed in Table 3 were administered to the 100 subjects immediately after the alcohol consumption. A positive effect was achieved by administration of the before and after compositions as evidenced by an improvement of hangover symptoms in the subjects within 12 hours of the administration of the first and second capsules.

The majority of the 100 subjects reported significant improvement in hangover symptoms with a range of alcohol consumption in both type and quantity. The subjects reported improvement in symptoms of fatigue, headache, nausea, dry mouth, sweating, anxiety, and dizziness. Many of the subjects also reported increased energy and alertness on the following day.

Two subjects reported temporary flushing including facial redness and a feeling of being hot. These symptoms lasted no more than 15 minutes. This is a very well-known side effect of niacin ingestion, although the composition of the disclosure has a much smaller dose of niacin than is typically reported to cause these symptoms. One of these subjects had undergone gastric bypass surgery, which results in unpredictable and rapid absorption of medications. This was presumed to be the cause of the flushing in that subject.

Several other symptoms were also reported including lucid dreams that were not unpleasant. This is a well-known result of B6 ingestion, although this effect is typically observed after the injection of much higher doses of B6 than the composition of the disclosure contains. A few subjects reported a feeling of calm or relaxation after consuming the composition of the disclosure.

While the improvement in energy was expected, over 30% of the subjects reported increased sleep quality and restfulness. This was a surprising result, and many subjects felt that this was a major reason that they had improved morning energy and motivation.

Additionally, several subjects having a genetic mutation that results in poor metabolism of alcohol, resulting in increased acetaldehyde accumulation, which causes facial flushing, reported delayed or absent alcohol flushing. This has not been reported by other hangover products with similar ingredients. The composition of the disclosure appears to promote acetaldehyde breakdown resulting in fewer symptoms of flushing.

Some subjects chose to take all four pills at bedtime after alcohol consumption, rather than taking two pills before alcohol consumption and two at bedtime after alcohol consumption. These subjects reported an improvement in symptoms. A few subjects forgot to take the product until waking up the next morning after alcohol consumption the night before and reported that the composition of the disclosure provided better relief of hangover symptoms than aspirin or ibuprofen when taken the following day after alcohol consumption the night before. Several subjects reported an improvement in mood and morning symptoms even with only the white or the green pills taken at bedtime based on user preference.

Approximately 20% of the subjects reported that taking the composition of the disclosure made them feel somewhat more sober after consuming alcohol.

There were no significant verifiable adverse events reported in the initial 100 subjects other than one possible incident of vomiting after taking the composition of the disclosure. However, vomiting is a common effect resulting from a hangover.

While the aforementioned method and composition have been described with regards to specific examples, it will be understood by those skilled in the art that minor variations and common substitutions may be applied without deviating from the scope of the disclosure.

What is claimed is:

1. A composition for alleviating alcohol consumption symptoms including hangover symptoms in a subject in need thereof, the composition comprising:
from about 33% to about 43% by weight of dihydromyricetin,
from about 27% to about 37% by weight of N-Acetyl Cysteine, and
soy lecithin.

2. The composition of claim 1, wherein the composition comprises from about 3% to about 5% by weight of soy lecithin.

3. The composition of claim 2, further comprising selenium, taurine, and salicin.

4. The composition of claim 1, wherein the soy lecithin is in powder form.

5. The composition of claim 4, further comprising niacin, vitamin B6, manganese, and prickly pear powder.

6. The composition of claim 2, further comprising:
from about 0.5% to about 1.5% by weight niacin,
from about 1% to about 3% by weight of vitamin B6,
from about 0.1% to about 0.2% by weight of manganese, and
from about 5% to about 7% by weight of prickly pear powder.

7. A composition for alleviating alcohol consumption symptoms including hangover symptoms in a subject in need thereof, the composition including a first composition and a separate second composition, the first composition comprising from about 20% to about 30% by weight of dihydromyricetin, from about 28% to about 38% by weight of N-Acetyl Cysteine, and soy lecithin, the second composition comprising from about 45% to about 55% by weight of dihydromyricetin from about 18% to about 28% by weight of N-Acetyl Cysteine, and soy lecithin.

8. The composition of claim 7, wherein the first composition further comprises from about 8% to about 18% by weight of prickly pear powder.

9. The composition of claim 7, wherein the second composition further comprises from about 5% to about 15% by weight of salicin.

10. A method of preparing the composition of claim 7, wherein the first composition and the second composition are in separate pill or capsule form.

11. A method of using the composition of claim 7 for alleviating alcohol consumption symptoms including hangover symptoms in a subject in need thereof.

12. The composition of claim 7, wherein the first composition comprises about 25% by weight of dihydromyricetin and about 33% by weight of N-Acetyl Cysteine, the second composition comprising about 49% by weight of dihydromyricetin and about 23% by weight of N-Acetyl Cysteine.

13. The composition of claim 7, wherein the first composition and the second composition are in separate pill or capsule form.

14. The composition of claim 8, wherein the first composition further comprises from about 4% to about 9% by weight of vitamin B6.

15. The composition of claim 14, wherein the first composition further comprises niacin, magnesium, zinc, selenium, manganese and taurine.

16. The composition of claim 9, wherein the second composition further comprises, zinc, selenium, manganese and taurine.

17. A method of alleviating alcohol consumption symptoms including hangover symptoms in a subject in need thereof, the method comprising:
administering a first composition comprising dihydromyricetin and N-Acetyl Cysteine to the subject prior to alcohol consumption and then administering a second composition comprising dihydromyricetin and N-Acetyl Cysteine to the subject after alcohol consumption.

18. The method according to claim 17, wherein the first composition comprises from about 20% to about 30% by weight of dihydromyricetin, from about 28% to about 38% by weight of N-Acetyl Cysteine, and soy lecithin, the second composition comprising from about 45% to about 55% by weight of dihydromyricetin, from about 18% to about 28% by weight of N-Acetyl Cysteine, and soy lecithin.

19. The method according to claim 18, wherein the first composition further comprises from about 8% to about 18% by weight of prickly pear powder.

20. The method according to claim 19, wherein the first composition further comprises from about 4% to about 9% by weight of Vitamin B6.

21. The method according to claim 20, wherein the first composition further comprises niacin, magnesium, zinc, selenium, manganese and taurine.

22. The method according to claim 20, wherein the second composition further comprises from about 5% to about 15% by weight of salicin.
